(19) United States
(12) Patent Application Publication
(10) Pub. No.: US 2022/0354782 A1
(43) Pub. Date: Nov. 10, 2022

(54) SUBCUTANEOUSLY INJECTABLE INSULIN AND GLUCAGON FORMULATIONS AND METHODS OF ADMINISTRATION

(71) Applicant: CASS PHARMACEUTICALS, INC., Franklin, TN (US)

(72) Inventors: Solomon S. Steiner, Franklin, TN (US); Lawrence D'Souza, San Diego, CA (US); Christopher A. Rhodes, San Diego, CA (US)

(21) Appl. No.: 17/760,596

(22) PCT Filed: Sep. 16, 2020
(86) PCT No.: PCT/US2020/051111

§ 371 Date: Mar. 15, 2022

Related U.S. Application Data
(60) Provisional application No. 62/901,408, filed on Sep. 17, 2019.

(51) Int. Cl. A61K 9/00; A61K 47/54; A61K 38/28; A61K 38/26; A61K 47/10; A61K 47/22

(52) U.S. Cl. CPC A61K 9/0019; A61K 47/545; A61K 38/28; A61K 38/26; A61K 47/10; A61K 47/22

(57) ABSTRACT

Provided are ultra fast-acting subcutaneously injectable insulin formulations as well as a stabilized subcutaneously injectable insulin and glucagon formulations, in addition to injection systems and methods of treatments and use thereof.

The present application claims the benefit of U.S. Provisional Application No. 62/901,408, filed Sep. 17, 2019, the entirety of which is incorporated by reference herein.

### BACKGROUND OF THE INVENTION

### Field of the Invention

The present invention is related to an ultra fast-acting subcutaneously injectable insulin solution, a stable insulin formulation and a stabilized subcutaneously injectable glucagon solution, as well as injection systems using one or both of those solutions and methods of treatment thereby.

### Related Background Art

The subcutaneous delivery of fast-acting insulin can be extremely important for effective treatment of diabetes mellitus, including Type 2 diabetes, Type 1 diabetes and gestational diabetes. Monomeric insulin, the active pharmacological form, has a molecular mass of 5808 Daltons. Insulin, however, that is produced and stored in the body exists as a hexameric form, from which monomers dissociate to become active. The hexamer form, due to its size, when injected into subcutaneous tissue is not readily absorbed and thus not fast acting. Insulin analogs have been developed by modifying amino acid sequences so as to reduce the stability of hexamer and dimer formation, making the monomer more readily available from the complex, leading to an increase in the subcutaneous absorption rate. Examples include HUMALOG® (insulin lispro) manufactured by Eli Lilly and Company, NOVOLOG® (insulin aspart) manufactured by Novo Nordisk Inc. and APIDRA® (insulin glulisine) manufactured by Sanofi-Aventis. Certain patients, however, have shown hypersensitivity to or other adverse effects to synthetic insulin analogs.

There exist fast-acting formulations which rely upon co-formulations of vasodilators to enhance absorption of the insulin by transient vasodilatory effect on the tissue in the subcutaneous space. Two products with vasodilators have been approved as commercial products, FIASP® and LYUMJEV®. FIASP (Novo Nordisk) contains niacinamide (vitamin B3) as a vasodilator and LYUMJEV (Eli Lilly) contains treprostinil as a vasodilator. In some patients, the vasodilators may cause side effects such as thrushing, including redness and itchiness in the hands and face.

Accordingly, there exists a need for fast-acting human recombinant insulin that is suitable for subcutaneous injection. In addition, synthetic insulin analogs still exist as hexamers, albeit, weaker hexamers. Thus, there exists a need for a solution of a synthetic insulin analog that becomes monomeric upon being exposed to the pH of the human interstitial fluid to provide an insulin that is absorbed into the blood in an extremely ultra-rapid fashion.

Subcutaneously injectable glucagon can also be extremely important to treat hypoglycemia, and particularly when used by a patient receiving insulin to treat diabetes mellitus that results in a hypoglycemic condition. However, glucagon has limited physical and chemical stability and is difficult to maintain in an aqueous solution in a stable form. Once in aqueous solution, the glucagon tends to form beta sheets, gels and loses potency, as well as chemically degrades (e.g. deamidation and oxidation), thus, rendering the solution impracticable for subcutaneous injection and subcutaneous infusion from pumps. The aqueous solution formulations of glucagon that have been approved (Eli Lilly Glucagon for Injection, Novo Nordisk GlucaGen HypoKit) require reconstitution prior to injection. In addition, the reconstituted aqueous formulations are stable for less than one day, and therefore these formulations cannot be used in infusion pumps, which require 3 to 7 day stability in the infusion pumps. Accordingly, there exists a need for a stabilized aqueous solution of glucagon that is suitable for subcutaneous injection and subcutaneous infusion from pumps.

### SUMMARY OF THE INVENTION

One aspect of the present invention pertains to an ultra fast-acting subcutaneously injectable insulin formulation comprising a solvated complex of a diketopiperazine and a monomeric insulin in an aqueous solution at a pH of about 6.0 to about 7.2.

Another aspect of the present invention pertains to an ultra fast-acting subcutaneously injectable insulin formulation comprising a solvated complex of a diketopiperazine and a monomeric insulin in an aqueous solution at a pH of about 6.0 to about 7.2; and one or more additional excipients.

Another aspect of the present invention pertains to an ultra fast-acting subcutaneously injectable insulin formulation comprising a monomeric insulin in an aqueous solution with one or more excipients, excluding a diketopiperazine, at a pH of about 6.0 to about 7.2.

Another aspect of the present invention pertains to a stabilized injectable insulin formulation comprising a monomeric insulin, with or without a diketopiperazine, and optionally one or more excipients, at a pH of about 6.0 to about 7.9, for instance 7.3 to 7.9.

Another aspect of the present invention pertains to a stabilized subcutaneously injectable glucagon formulation comprising a solvated complex of a diketopiperazine and glucagon in an aqueous solution at a pH of about 6.4 to about 7.9.

Another aspect of the present invention pertains to a stabilized subcutaneously injectable glucagon formulation comprising a solvated complex of a diketopiperazine and glucagon in an aqueous solution at a pH of about 6.4 to about 7.9; and one or more additional excipients.

Another aspect of the present invention pertains to a stabilized subcutaneously injectable glucagon formulation comprising glucagon in an aqueous solution with one or more excipients, excluding a diketopiperazine, at a pH of about 6.4 to about 7.9.

Another aspect of the present invention pertains to an injection system for subcutaneous injection of insulin comprising a reservoir communicating with one or more needles for subcutaneous injection to a patient in need thereof, wherein said reservoir contains a therapeutically effective amount of the ultra fast-acting subcutaneously injectable insulin solution of the invention. The invention also provides an injection system for subcutaneous injection of glucagon comprising a reservoir communicating with one or more needles for subcutaneous injection to a patient in need thereof, wherein said reservoir contains a therapeutically effective amount of the stabilized subcutaneously injectable glucagon solution of the invention.

Another aspect of the present invention pertains to pen and pump devices with monitors for dose number and dose quantity to increase patient compliance, and for connection of these analytical measures to smart phone, tablet, or electronic records systems based on applications for patient and physician monitoring of therapy.

Another aspect of the present invention pertains to a bimodal injection system for subcutaneous injection of insulin or glucagon as needed by a patient, the system comprising a first reservoir and a second reservoir, each communicating with one or more needles for subcutaneous injection to a patient in need thereof, wherein the first reservoir contains a therapeutically effective amount of the ultra fast-acting subcutaneously injectable insulin solution of the invention and the second reservoir contains a therapeutically effective amount of the stabilized subcutaneously injectable glucagon solution of the invention.

Another aspect of the present invention pertains to a method of treating a patient in need of insulin comprising the step of subcutaneously administering to the patient in need thereof a therapeutically effective amount of the ultra fast-acting subcutaneously injectable insulin solution of the invention. In yet another embodiment, a method is disclosed of treating a patient in need of glucagon comprising the step of subcutaneously administering to the patient in need thereof a therapeutically effective amount of the stabilized subcutaneously injectable glucagon solution of the invention.

Another aspect of the present invention pertains to methods of manufacturing the ultra fast-acting subcutaneously injectable insulin solution of the invention and the stabilized subcutaneously injectable glucagon solution of the invention.

Yet another aspect of the present invention pertains to the use of the ultra fast-acting subcutaneously injectable insulin solution of the invention and/or the stabilized subcutaneously injectable glucagon solution of the invention for the treatment of, or for the preparation of a medicament for the treatment of, diabetes mellitus, including Type 2 diabetes, Type 1 diabetes and gestational diabetes and/or hypoglycemia.

### BRIEF DESCRIPTIONS OF THE DRAWINGS

FIG. 1 depicts the glucose response from subcutaneous injections of a formulation in accordance with the present invention compared to a standard lispro insulin as described in Example 8.

FIG. 2 depicts the glucose response from subcutaneous injections of a formulation in accordance with the present invention at pH 6.5 compared to a formulation in accordance with the present invention at pH 7.5 as described in Example 8.

FIG. 3 is a graph depicting the concentration of FDKP for forming micelles in phosphate buffer at pH 7.4 above 0.03 mg/ml.

### DETAILED DESCRIPTION OF THE INVENTION

The present invention provides insulin formulations that make it possible for insulin to be present in its monomeric form upon being exposed to the pH of the human interstitial fluid via, for example, subcutaneous injection, to provide an insulin that is absorbed into the blood in an extremely ultra-rapid fashion. The present invention also provides glucagon formulations that allow glucagon to remain stable, for instance retain stability for at least 3 to 7 days, so that these formulations are suitable, for instance, for subcutaneous injection and subcutaneous infusion from pumps.

The present invention includes ultra fast-acting subcutaneously injectable formulations comprising insulin. In an embodiment, the ultra fast-acting subcutaneously injectable formulation comprises a solvated complex of a diketopiperazine and monomeric insulin in an aqueous solution at a pH of about 6.0 to about 7.2, or about 6.0 to about 7.0, or about 6.0 to about 6.9, or about 6.1 to about 6.9, or about 6.1 to about 6.8, or about 6.4 to about 6.8, or about 6.4 to about 6.6. In an embodiment, the pH is about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or any amount in between. In an embodiment, the aqueous solution is devoid of one or more additional excipients. In an embodiment, the aqueous solution is water, optionally containing sodium chloride.

In an embodiment, the ultra fast-acting subcutaneously injectable formulation comprises a solvated complex of a diketopiperazine and monomeric insulin in an aqueous solution with one or more additional excipients at a pH of about 6.0 to about 7.2, or about 6.0 to about 7.0, or about 6.0 to about 6.9, or about 6.1 to about 6.9, or about 6.1 to about 6.8, or about 6.4 to about 6.8. In an embodiment, the pH is about 6.0, or about 6.1, or about 6.2, or about 6.3, or about 6.4 or about 6.5, or about 6.6, or about 6.7, or about 6.8, or about 6.9, or about 7.0, or about 7.1, or about 7.2, or any amount in between.

In an embodiment, the ultra fast-acting subcutaneously injectable formulation comprises insulin in an aqueous solution with one or more excipients, excluding a diketopiperazine, at a pH of about 6.0 to about 7.2, or about 6.0 to about 7.0, or about 6.0 to about 6.9, or about 6.1 to about 6.9, or about 6.1 to about 6.8, or about 6.4 to about 6.8. In an embodiment, the pH is about 6.0, or about 6.1, or about 6.2, or about 6.3, or about 6.4 or about 6.5, or about 6.6, or about 6.7, or about 6.8, or about 6.9, or about 7.0, or about 7.1, or about 7.2, or any amount in between.

As used herein, insulin includes human and animal sourced insulin, synthetic insulin or recombinant human insulin such as rapid-acting insulin analogs, such as lispro, insulin aspart, and insulin glulisine. Preferably, the insulin is recombinant human insulin or synthetic insulin. Most preferably the insulin is rapid-acting insulin analogs such as lispro, aspart, or glulisine where such rapid-acting insulin analogs becomes monomeric upon being exposed to the pH of the human interstitial fluid to provide an insulin that is absorbed into the blood in an extremely ultra-rapid fashion.

In an embodiment, the formulation includes about 1 mg/ml to about 20 mg/ml, or about 1 mg/ml to about 15 mg/ml, or about 1 mg/ml to about 10 mg/ml, or about 1 mg/ml to about 5 mg/ml, or about 1 mg/ml to about 4.5 mg/ml, or about 2 mg/ml to about 20 mg/ml, or about 2 mg/ml to about 15 mg/ml, or about 2 mg/ml to about 10 mg/ml, or about 2 mg/ml to about 5 mg/ml, or about 2 mg/ml to about 4.5 mg/ml, or about 3 mg/ml to about 20 mg/ml, or about 3 mg/ml to about 15 mg/ml, or about 3 mg/ml to about 10 mg/ml, or about 3 mg/ml to about 5 mg/ml, or about 3 mg/ml to about 4.5 mg/ml, or about 3.5 mg/ml to about 20 mg/ml, or about 3.5 mg/ml to about 15 mg/ml, or about 3.5 mg/ml to about 10 mg/ml, or about 3.5 mg/ml to about 5 mg/ml, or about 3.75 mg/ml to about 4.75 mg/ml, or about 4 mg/ml to about 20 mg/ml, or about 4 mg/ml to about 15 mg/ml, or about 4 mg/ml to about 10 mg/ml, or about 4 mg/ml to about 5 mg/ml, or about 4.0 mg/ml to about 4.5 mg/ml of monomeric insulin, preferably from a monomeric insulin, for example a recombinant human insulin or rapid acting insulin analog described herein. In an embodiment, the formulation includes about 3 mg/ml, or about 3.25 mg/ml, or about 3.5 mg/ml, or about 3.75 mg/ml, or about 4 mg/ml, or about 4.1 mg/ml, or about 4.2 mg/ml, or about 4.3 mg/ml, or about 4.4 mg/ml, or about 4.5 mg/ml, or about 4.6 mg/ml, or about 4.75 mg/ml, or about 5 mg/ml, or about 5.25 mg/ml, or about 5.5 mg/ml, or about 5.75 mg/ml, or about 6 mg/ml, or about 6.5 mg/ml, or about 7 mg/ml, or about 8 mg/ml, or about 9 mg/ml, or about 10 mg/ml or about 11 mg/ml or about 12 mg/ml, or about 13 mg/ml, or about 14 mg/ml or about 15 mg/ml or about 16 mg/ml, or about 17 mg/ml, or about 18 mg/ml or about 19 mg/ml, or about 20 mg/ml. or any amount in between, of monomeric insulin, preferably from a recombinant human insulin or rapid acting insulin analog described herein.

As used herein, ultra fast-acting means that the formulation includes a rapid-acting insulin analog described herein, where such rapid-acting insulin analog becomes monomeric upon being exposed to the pH of the human interstitial fluid to provide an insulin that is absorbed into the blood in an extremely ultra-rapid fashion.

As used herein, a complex of diketopiperazine and insulin or diketopiperazine and glucagon refers to an intermolecular interaction between insulin and glucagon and diketopiperazine such as, for example, hydrogen bonding. An effect of these interactions was observed, for instance, by comparing solubility of insulin as described in Example 9.

As used herein, excluding a diketopiperazine means that the formulation does not include a complex of diketopiperazine and insulin or a complex of diketopiperazine and glucagon. A diketopiperazine may still be optionally present in the formulation, albeit not in the aforementioned complex form.

The present invention also includes a stabilized subcutaneously injectable formulation comprising glucagon. In an embodiment, the stabilized subcutaneously injectable formulation comprises a solvated complex of a diketopiperazine and glucagon in an aqueous solution at a pH of about 6.4 to about 8.0, or about 6.4 to about 6.8 or about 7.0 to about 7.9, or about 7.1 to about 7.9, or about 7.3 to about 7.9, or about 7.3 to about 7.8, or about 7.6 to about 7.9. In an embodiment, the pH is about 6.4, or about 6.5, or about 6.6, or about 6.7, or about 6.8, or about 6.9 about 7.0, or about 7.1, or about 7.2, or about 7.3, or about 7.4, or about 7.5, or about 7.6, or about 7.7, or about 7.8, or about 7.9, or about 8.0, or any amount in between. In an embodiment, the aqueous solution is devoid of one or more additional excipients. In an embodiment, the aqueous solution is water, optionally containing sodium chloride.

In an embodiment, the stabilized subcutaneously injectable formulation comprises a solvated complex of a diketopiperazine and glucagon in an aqueous solution with one or more additional excipients at a pH of about 6.4 to about 8.0, or about 6.4 to about 6.8 or about 7.0 to about 7.9, or about 7.1 to about 7.9, or about 7.3 to about 7.9, or about 7.3 to about 7.8, or about 7.6 to about 7.9. In an embodiment, the pH is about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0, or any amount in between.

In an embodiment, the stabilized subcutaneously injectable formulation comprises glucagon in various aqueous solutions with one or more excipients, excluding a diketopiperazine, at a pH of about 6.4 to about 8.0, or about 6.4 to about 6.8, or about 7.0 to about 7.9, or about 7.1 to about 7.9, or about 7.3 to about 7.9, or about 7.3 to about 7.8, or about 7.6 to about 7.9. In an embodiment, the pH is about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0, or any amount in between.

In an embodiment, the stabilized subcutaneously injectable formulation comprises glucagon in amount of about 0.1 mg/ml to about 5 mg/ml, or about 0.1 mg/ml to about 4 mg/ml, or about 0.1 mg/ml to about 3 mg/ml, or about 0.1 mg/ml to about 2 mg/ml, or about 0.1 mg/ml to about 1 mg/ml, or about 0.1 mg/ml to about 0.8 mg/ml, or about 0.2 mg/ml to about 4 mg/ml, or about 0.2 mg/ml to about 3 mg/ml, or about 0.2 mg/ml to about 2 mg/ml, or about 0.2 mg/ml to about 1 mg/ml, or about 0.2 mg/ml to about 0.8 mg/ml, or about 0.2 mg/ml to about 0.7 mg/ml, or about 0.3 mg/ml to about 4 mg/ml, or about 0.3 mg/ml to about 3 mg/ml, or about 0.3 mg/ml to about 2 mg/ml, about 0.3 mg/ml to about 1.2 mg/ml, or about 0.3 mg/ml to about 1 mg/ml, or about 0.3 mg/ml to about 0.8 mg/ml, or about 0.3 mg/ml to about 0.7 mg/ml, or about 0.3 mg/ml to about 0.6 mg/ml, or about 0.4 mg/ml to about 0.6 mg/ml. In an embodiment, the stabilized subcutaneously injectable formulation comprises glucagon in amount of about 0.1 mg/ml, or about 0.2 mg/ml, or about 0.3 mg/ml, or about 0.4 mg/ml, or about 0.5 mg/ml, or about 0.6 mg/ml, or about 0.7 mg/ml, or about 0.8 mg/ml, or about 0.9 mg/ml, or about 1.0 mg/ml, or about 1.25 mg/ml, or about 1.5 mg/ml, or about 1.75 mg/ml, or about 2 mg/ml, or about 2.5 mg/ml, or about 3 mg/ml, or about 3.5 mg/ml or about 4 mg/ml, or about 4.5 mg/ml, or about 5 mg/ml, or any amount in between.

In accordance with the present invention, the glucagon formulations are chemically stable (lack of degradation by either deamidation and/or oxidation or hydrolysis) and physically stable (do not form beta sheets, fibrils, precipitates, aggregates) in an aqueous solution and suitable for subcutaneous injection and subcutaneous infusion from pumps. That stability, as discussed in more detail below, is preferably maintained for at least 3 to 7 days.

In an embodiment, the diketopiperazine (“DKP”) is a 2,5 diketopiperazine represented by formula (I):

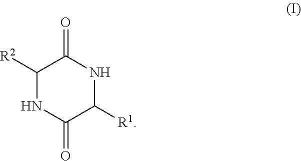

In formula (I), R¹ and R² are independently X—Y—Z—W, and wherein X is a C1-C20 straight or branched or cyclic alkyl, aralkyl, alkaryl, alkenyl, alkynyl, heteroalkyl, heterocyclic, alkyl-heterocyclic or heterocyclic-alkyl; Y may be present or absent and when present is —C(O)O, —OC(O), C(O)NH, —NH, —NX, —OXO, —O, —NHC(O), —OP(O), —P(O)O, —OP(O)₂, —P(O)₂O, —OS(O)₂, or —S(O)₃; Z may be present or absent and when present is a C1-C20 straight or branched or cyclic alkyl, aralkyl, alkaryl, alkenyl, alkynyl, heteroalkyl, heterocyclic, alkyl-heterocyclic or heterocyclic-alkyl; and W is an acid group.

Alkyl, as used herein, is C1-C20 straight or branched chain or cyclic group, preferably C1-C12 straight or branched chain, and most preferably C1-C6 straight or branched chain. Alkenyl, as used herein, is C2-C20 straight or branched chain with at least one double bond, preferably C2-C12 straight or branched chain with at least one double bond and more preferably C2-C6 straight or branch chain with a least one double bond. Alkynyl, as used herein, is C2-C20 straight or branched chain with at least one triple bond, preferably C2-C12 straight or branched chain with at least one triple bond and more preferably C2-C6 straight or branch chain with a least one triple bond. Aralkyl and alkaryl, as used herein, is an aromatic group in combination with an alkyl group, wherein alkyl is as defined above and aromatic is a 5-, 6-, or 7-aromatic ring. Heteroalkyl, as used herein, is 1 to 4 heteroatoms substituted on an alkyl as described above. Exemplary heteroatoms include N, O, S and P. Heterocyclic, as used herein, is a 4 to 7 membered ring having 1 to 4 hetero atoms in the ring. Alkyl-heterocyclic and heterocyclic-alky, as used herein, are a combination of an alkyl group as defined herein and heterocyclic group as defined herein.

The acid group W is preferably selected from the group consisting of cis —CH═CH—CO₂H, trans —CH═CH—CO₂H, —CH(CH₃)═CH(CH₃)—CO₂H, —(CH₂)₃—CO₂H, —CH₂CH(CH₃)—CO₂H, —CH(CH₂CO₂H)═CH₂, -(tetrafluoro)benzoic acid, -benzoic acid and —CH(NHC(O)CF₃)—CH₂—CO₂H.

In an embodiment, the diketopiperazine may be 2,5-diketo-3,6-di(4-succinylaminobutyl)piperazine (“Succinic Acid DKP”), 2,5-diketo-3,6-di(4-fumarylaminobutyl)piperazine (“FDKP”), 2,5-diketo-3,6-di(4-maleylaminobutyl)piperazine, or 2,5-diketo-3,6-di(4-glutarylaminobutyl)piperazine. Preferably, the diketopiperazine is FDKP. In an embodiment, disodium FDKP may be used in the preparation of glucagon and insulin formulations by adding the solid disodium FDKP to insulin or glucagon in the various buffer, co-solvent, and excipient formulations as described herein, and adjusting the pH up or down to the desired pH, described herein, with hydrochloric acid or sodium hydroxide or the like to achieve the final clear solution formulation.

The preparation of the diketopiperazines used herein is well understood by those skilled in the art. For example, U.S. Pat. No. 6,071,497, which is incorporated by reference in its entirety herein, discusses in detail methods of synthesis of diketopiperazines.

In an embodiment, insulin formulations described herein may include about 1 mg/ml to about 180 mg/ml, or about 1 mg/ml to about 150 mg/ml, or about 1 mg/ml to about 120 mg/ml, or about 1 mg/ml to about 100 mg/ml, or about 1 mg/ml to about 80 mg/ml, or about 1 mg/ml to about 50 mg/ml, or about 1 mg/ml to about 45 mg/ml, or about 1 mg/ml to about 40 mg/ml, or about 1 mg/ml to about 38 mg/ml, or about 1 mg/ml to about 36 mg/ml, or about 1 mg/ml to about 35 mg/ml, or about 1 mg/ml to about 30 mg/ml, or about 1 mg/ml to about 25 mg/ml, or about 2 mg/ml to about 40 mg/ml, or about 5 mg/ml to about 40 mg/ml, or about 10 mg/ml to about 40 mg/ml, or about 15 mg/ml to about 40 mg/ml, or about 20 mg/ml to about 40 mg/ml, or about 25 mg/ml to about 40 mg/ml, or about 30 mg/ml to about 180 mg/ml, or about 30 mg/ml to about 150 mg/ml, or about 30 mg/ml to about 120 mg/ml, or about 30 mg/ml to about 100 mg/ml, or about 30 mg/ml to about 80 mg/ml, or about 30 mg/ml to about 50 mg/ml, or about 30 mg/ml to about 40 mg/ml, or about 35 mg/ml to about 180 mg/ml, or about 35 mg/ml to about 150 mg/ml, or about 35 mg/ml to about 120 mg/ml, or about 35 mg/ml to about 100 mg/ml, or about 35 mg/ml to about 80 mg/ml, or about 35 mg/ml to about 50 mg/ml, or about 35 mg/ml to about 40 mg/ml, of a diketopiperazine, preferably FDKP. In an embodiment, insulin formulations may include about 2.0 mg/ml, or about 2.5 mg/ml, or about 3.0 mg/ml, or about 3.5 mg/ml, or about 4.0 mg/ml, or about 4.5 mg/ml, or about 5.0 mg/ml, or about 5.5 mg/ml, or about 6.0 mg/ml, or about or about 6.5 mg/ml, or about or about 7.0 mg/ml, or about 7.5 mg/ml, or about 8.0 mg/ml, or about 8.5 mg/ml, or about 9.0 mg/ml, or about 9.5 mg/ml, or about 10 mg/ml, or about 10.5 mg/ml, or about 11 mg/ml, or about 11.5 mg/ml, or about 12 mg/ml, or about 12.5 mg/ml, or about 13 mg/ml, or about 13.5 mg/ml, or about 14 mg/ml, or about 14.5 mg/ml, or about 15 mg/ml, or about 20 mg/ml, or about 22.5 mg/ml, or about 25 mg/ml, or about 27.5 mg/ml, or about 30 mg/ml, or about 31 mg/ml, or about 32 mg/ml, or about 33 mg/ml, or about 34 mg/ml, or about 35 mg/ml, or about 36 mg/ml, or about 37 mg/ml, or about 38 mg/ml, or about 39 mg/ml, or about 40 mg/ml, or about 50 mg/ml, or about 60 mg/ml or about 70 mg/ml, or about 80 mg/ml or about 90 mg/ml, or about 100 mg/ml, or about 110 mg/ml, or about 120 mg/ml, or about 130 mg/ml, or about 140 mg/ml, or about 150 mg/ml, or about 160 mg/ml, or about 170 mg/ml, or about 180 mg/ml, or any amount in between, of a diketopiperazine, preferably FDKP.

In an embodiment, the ratio of insulin to diketopiperazine, preferably FDKP, in weight is about 1 to about 15, or about 1 to about 14, or about 1 to about 13, or about 1 to about 12, or about 1 to about 11, or about 1 to about 10, or about 1 to about 9, or about 1 to about 8, or about 1 to about 7, or about 1 to about 6, or about 1 to about 5, or about 1 to about 4, or about 1 to about 3, or any ratio therebetween, for instance from about 1 to about 15 to about 1 to about 3. Preferably, the ratio of insulin to diketopiperazine, preferably FDKP, in weight is from about 1 to about 8 (about 1:8) to about 1 to about 10 (about 1:10), such as about 1 to about 9 (about 1:9).

In an embodiment, glucagon formulations described herein may include about 2 mg/ml to about 15 mg/ml, or about 2.5 mg/ml to about 15 mg/ml, or about 3.0 mg/ml to about 15 mg/ml, or about 3.5 mg/ml to about 15 mg/ml, or about 4.0 mg/ml mg/ml to about 15 mg/ml, or about 4.5 mg/ml to about 15 mg/ml, or about 5 mg/ml to about 15 mg/ml, or about 5.5 mg/ml to about 15 mg/ml, or about 6 mg/ml to about 15 mg/ml, about 6.5 mg/ml to about 15 mg/ml, about 7 mg/ml to about 15 mg/ml, about 7.5 mg/ml to about 15 mg/ml, about 8 mg/ml to about 15 mg/ml, about 8.5 mg/ml to about 15 mg/ml, about 9 mg/ml to about 15 mg/ml, or about 9.5 mg/ml to about 15 mg/ml, or about 10 mg/ml to about 15 mg/ml, or about 2 mg/ml to about 10 mg/ml, or about 2.5 mg/ml to about 10 mg/ml, or about 3.0 mg/ml to about 10 mg/ml, or about 3.5 mg/ml to about 10 mg/ml, or about 4.0 mg/ml mg/ml to about 10 mg/ml, or about 4.5 mg/ml to about 10 mg/ml, or about 5 mg/ml to about 10 mg/ml, or about 5.5 mg/ml to about 10 mg/ml, or about 6 mg/ml to about 10 mg/ml, about 6.5 mg/ml to about 10 mg/ml, about 7 mg/ml to about 10 mg/ml, about 7.5 mg/ml to about 10 mg/ml, about 8 mg/ml to about 10 mg/ml, about 8.5 mg/ml to about 10 mg/ml, about 9 mg/ml to about 10 mg/ml, or about 9.5 mg/ml to about 10 mg/ml, of a diketopiperazine, preferably FDKP. In an embodiment, glucagon formulations may include about 2.0 mg/ml, or about 2.5 mg/ml, or about 3.0 mg/ml, or about 3.5 mg/ml, or about 4.0 mg/ml, or about 4.5 mg/ml, or about 5.0 mg/ml, or about 5.5 mg/ml, or about 6.0 mg/ml, or about or about 6.5 mg/ml, or about or about 7.0 mg/ml, or about 7.5 mg/ml, or about 8.0 mg/ml, or about 8.5 mg/ml, or about 9.0 mg/ml, or about 9.5 mg/ml, or about 10 mg/ml, or about 10.5 mg/ml, or about 11 mg/ml, or about 11.5 mg/ml, or about 12 mg/ml, or about 12.5 mg/ml, or about 13 mg/ml, or about 13.5 mg/ml, or about 14 mg/ml, or about 14.5 mg/ml, or about 15 mg/ml, any amount in between, of a diketopiperazine, preferably FDKP.

In an embodiment, the ratio of glucagon to diketopiperazine, preferably FDKP, in weight is about 1 to about 100, or about 1 to about 90, or about 1 to about 80, or about 1 to about 70, or about 1 to about 60, or about 1 to about 50, or about 1 to about 40, or about 1 to about 35, or about 1 to about 30, or about 1 to about 25, or about 1 to about 20, or about 1 to about 15, or about 1 to about 14, or about 1 to about 13, or about 1 to about 12, or about 1 to about 11, or about 1 to about 10, or about 1 to about 9, or about 1 to about 8, or about 1 to about 7, or about 1 to about 6, or about 1 to about 5, or about 1 to about 4, or about 1 to about 3, or any ratio therebetween, for instance from about 1 to about 100 (about (1:100) to about 1 to about 3 (about 1:3). Preferably, the ratio of glucagon to diketopiperazine, preferably FDKP, in weight is from about 1 to about 10 (about 1:10) to about 1 to about 20 (about 1:20).

In the present invention, the one or more excipients in the formulations include standard pharmaceutical excipients and generally recognized as safe (“GRAS”) excipients used for injectable products, in the class of buffers, solubilizers, aggregation preventers, surfactants, absorption enhancers and permeation enhancers, metal chelators, preservatives, tonicity modifiers, vasodilators, sugars, dextran molecules and others known in the art.

Buffer excipients include buffers such as tris(hydroxymethyl)aminomethane (“TRIS”), phosphate, phosphate buffered saline, arginine, glycine, phosphate-citrate, histidine, In an embodiment, the buffer is a phosphate or phosphate buffered saline. In an embodiment, about 1 mM to about 100 mM, or about 1 mM to about 50 mM, or about 1 mM to about 25 mM, or about 1 mM to about 15 mM, or about 1 mM to about 10 mM, or about 5 mM to about 100 mM, or about 5 mM to about 50 mM, or about 5 mM to about 25 mM, or about 5 mM to about 15 mM, or about 5 mM to about 10 mM, or about 10 mM to about 100 mM, or about 10 mM to about 50 mM, or about 10 mM to about 25 mM, or about 10 mM to about 15 mM, of a buffer is used in the formulations described herein.

Solubilizing excipients include excipients such as dimethyl sulfoxide (“DMSO”), N-Methyl-2-pyrrolidone (“NMP”), ethanol, propylene glycol, glycerol, polyethylene glycol, including but not limited to PEG 300, PEG 400, and PEG 4000, with NMP as a preferred solubilizing excipient. These solubilizing excipients may be used as excipients or co-solvents at acceptable levels for injections such as subcutaneous, intramuscular, and/or intradermal. In an embodiment, formulations may include about 0.5 v/v % to about 25 v/v %, or about 1 v/v % to about 25 v/v %, or about 5 v/v % to about 25 v/v %, or about 6 v/v % to about 25 v/v %, or about 7 v/v % to about 25 v/v %, or about 8 v/v % to about 25 v/v %, or about 9 v/v % to about 25 v/v %, or about 10 v/v % to about 25 v/v %, or about 11 v/v % to about 25 v/v %, or about 12 v/v % to about 25 v/v %, or about 13 v/v % to about 25 v/v %, or about 14 v/v % to about 25 v/v %, or about 15 v/v % to about 25 v/v %, or about 16 v/v % to about 25 v/v %, or about 17 v/v % to about 25 v/v %, or about 18 v/v % to about 25 v/v %, or about 19 v/v % to about 25 v/v %, or about 20 v/v % to about 25 v/v %, or 0.5 v/v % to about 20 v/v %, or about 1 v/v % to about 20 v/v %, or about 5 v/v % to about 20 v/v %, or about 6 v/v % to about 20 v/v %, or about 7 v/v % to about 20 v/v %, or about 8 v/v % to about 20 v/v %, or about 9 v/v % to about 20 v/v %, or about 10 v/v % to about 20 v/v %, or about 11 v/v % to about 20 v/v %, or about 12 v/v % to about 20 v/v %, or about 13 v/v % to about 20 v/v %, or about 14 v/v % to about 20 v/v %, or about 15 v/v % to about 20 v/v %, or about 16 v/v % to about 20 v/v %, or about 17 v/v % to about 20 v/v %, or about 18 v/v % to about 20 v/v %, or 0.5 v/v % to about 15 v/v %, or about 1 v/v % to about 15 v/v %, or about 5 v/v % to about 15 v/v %, or about 6 v/v % to about 15 v/v %, or about 7 v/v % to about 15 v/v %, or about 8 v/v % to about 15 v/v %, or about 9 v/v % to about 15 v/v %, or about 10 v/v % to about 15 v/v %, or about 11 v/v % to about 15 v/v %, or about 12 v/v % to about 15 v/v %, or about 13 v/v % to about 15 v/v % of the solubilizing excipient. In an embodiment, formulations may include about 0.5 v/v %, or about 1 v/v %, or about 2.5 v/v %, or about 5 v/v %, or about 6 v/v % or about 7 v/v %, or about 8 v/v %, or about 9 v/v %, or about 10 v/v %, or about 11 v/v %, or about 12 v/v %, or about 13 v/v %, or about 14 v/v %, or about 15 v/v %, or about 16 v/v %, or about 17 v/v %, or about 18 v/v %, or about 19 v/v % or about 20 v/v %, or about 21 v/v %, or about 22 v/v %, or about 23 v/v %, or about 24 v/v %, or about 25 v/v % of the solubilizing excipient, and any amount in between.

Aggregation preventers include excipients such as phenol, nicotinamide, nicotinic acid, arginine, glycine, and other amino acids, all charged organic molecules with hydrophobic moieties interacting with charges and hydrophobic sites on insulin and glucagon. In an embodiment, formulations may include about 0.01 mM to about 10 mM, or about 0.01 mM to about 5 mM, or about 0.01 mM to about 2 mM, or about 0.01 mM to about 1.5 mM, or about 0.01 mM to about 1 mM, or about 1 mM to about 100 mM, or about 1 mM to about 50 mM, or about 1 mM to about 25 mM, or about 1 mM to about 15 mM, or about 1 mM to about 10 mM, or about 5 mM to about 100 mM, or about 5 mM to about 50 mM, or about 5 mM to about 25 mM, or about 5 mM to about 15 mM, or about 5 mM to about 10 mM, or about 10 mM to about 100 mM, or about 10 mM to about 50 mM, or about 10 mM to about 25 mM, of an aggregation preventor.

Surfactants include excipients such as polysorbates, alkylglycosides, ionic and non-ionic surfactants, bolaamphiphile surfactants with a hydrophobic core containing highly water soluble and symmetrical or unsymmetrical end groups on either side of the hydrophobic core, for example FDKP. In an embodiment, formulations may include about 0.01 v/v % to about 20 v/v %, about 0.01 v/v % to about 18 v/v %, about 0.01 v/v % to about 15 v/v %, about 0.01 v/v % to about 12 v/v %, about 0.01 v/v % to about 10 v/v %, or about 0.01 v/v % to about 5 v/v %, or about 0.01 v/v % to about 2 v/v %, or about 0.01 v/v % to about 1.5 v/v %, or about 0.01 v/v % to about 1 v/v %, of a surfactant.

Absorption enhancers and permeation enhancers include excipients such as polysorbates, glycocolates, glycholic acid, citric acid, ethylenediaminetetraacetic acid (“EDTA”), methyl beta cyclodextrin and other cyclodextrins, dipalmitoylphosphatidylcholine (“DDPC”), polyamidoamine (“PAMAM”) dendrimers, and organic compounds with hydrophobic and charged portions, for example, FDKP. In an embodiment, formulations may include about 1 mM to about 100 mM, or about 1 mM to about 50 mM, or about 1 mM to about 25 mM, or about 1 mM to about 15 mM, or about 1 mM to about 10 mM, or about 5 mM to about 100 mM, or about 5 mM to about 50 mM, or about 5 mM to about 25 mM, or about 5 mM to about 15 mM, or about 5 mM to about 10 mM, or about 10 mM to about 100 mM, or about 10 mM to about 50 mM, or about 10 mM to about 25 mM, of an absorption enhancer and/or permeation enhancer.

Metal chelators include excipients such as EDTA, citric acid, salicylic acid, histidine, and amino acids, which bind metals through charge or polar groups, including FDKP. In an embodiment, formulations may include about 1 mM to about 100 mM, or about 1 mM to about 50 mM, or about 1 mM to about 25 mM, or about 1 mM to about 15 mM, or about 1 mM to about 10 mM, or about 5 mM to about 100 mM, or about 5 mM to about 50 mM, or about 5 mM to about 25 mM, or about 5 mM to about 15 mM, or about 5 mM to about 10 mM, or about 10 mM to about 100 mM, or about 10 mM to about 50 mM, or about 10 mM to about 25 mM, of a metal chelator.

Preservatives include excipients such as phenol, m-cresol, benzyl alcohol, parabens and parabens esters, phenoxyethanol, benzalkonium chloride, for which phenol and m-cresol are preferred preservatives. In an embodiment, the formulation may include about 1 mg/ml to about 10 mg/ml, or about 2 mg/ml to about 8 mg/ml, or about 2 mg/ml to about 4 mg/ml, or about 2.5 mg/ml to about 7 mg/ml, or about 2.5 mg/ml to about 5 mg/ml, or about 2.5 mg/ml to about 4 mg/ml, or about 2.5 mg/ml to about 3.5 mg/ml, or about 2.75 mg/ml to about 3.25 mg/ml, or about 2.9 mg/ml to about 3.15 mg/ml, of a preservative. In an embodiment, formulation may include about 1 mg/ml, or about 1.5 mg/ml, or about 2 mg/ml, or about 2.5 mg/ml, or about 2.75 mg/ml, or about 2.8 mg/ml, or about 2.9 mg/ml, or about 3 mg/ml, or about 3.1 mg/ml, or about 3.15 mg/ml, or about 3.2 mg/ml, or about 3.25 mg/ml, or about 3.3 mg/ml, about 3.35 mg/ml, or about 3.5 mg/ml, or about 4 mg/ml, or about 4.5 mg/ml, or about 5 mg/ml, or about 5.5 mg/ml, or about 6 mg/ml, or about 6.5 mg/ml, or about 7 mg/ml, or about 7.5 mg/ml, or about 8 mg/ml, or about 8.5 mg/ml, or about 9 mg/ml, or about 9.5 mg/ml or about 10 mg/ml of a preservative, and any amount in between. In an embodiment, the preservative at any of the above amounts is m-cresol. For instance, the formulation includes about 3 mg/ml of m-cresol.

In an embodiment, the formulation may include about 0.01 v/v % to about 10 v/v %, or about 0.01 v/v % to about 8 v/v %, or about 0.01 v/v % to about 6 v/v %, about 0.01 v/v % to about 5 v/v %, about 0.01 v/v % to about 4 v/v %, about 0.01 v/v % to about 3 v/v %, about 0.01 v/v % to about 2 v/v %, about 0.01 v/v % to about 1 v/v %, or about 1 v/v % to about 10 v/v %, or about 1 v/v % to about 8 v/v %, or about 1 v/v % to about 6 v/v %, about 1 v/v % to about 5 v/v %, about 1 v/v % to about 4 v/v %, about 1 v/v % to about 3 v/v %, about 1 v/v % to about 2 v/v % of a preservative. In an embodiment, the formulation may include about 0.01 v/v %, or about 1 v/v %, or about 2 v/v %, or about 3 v/v %, or about 3.5 v/v %, or about 4 v/v %, or about 4.5 v/v %, or about 5 v/v %, or about 6 v/v %, or about 7 v/v %, or about 8 v/v %, or about 9 v/v %, or about 10 v/v %, of a preservative, and any amount in between. For instance, the formulation includes about 3 mg/ml of benzyl alcohol.

In an embodiment, the formulation may include about 0.001 w/v % to about 2 w/v %, or about 0.001 w/v % to about 1.5 w/v % or about 0.001 w/v % to about 1 w/v %, or about 0.001 w/v % to about 0.5 w/v %, or about 0.001 w/v % to about 0.1 w/v %, or about 0.01 w/v % to about 2 w/v %, or about 0.01 w/v % to about 1.5 w/v %, about 0.01 w/v % to about 1 w/v %, about 0.01 w/v % to about 0.5 w/v %, about 0.01 w/v % to about 0.1 w/v %, or about 0.02 w/v % to about 0.08 w/v %, or about 0.05 w/v % to about 0.075 w/v %. In an embodiment, the formulation may include about 0.001 w/v %, or about 0.01 w/v % or about 0.02 w/v % or about 0.03 w/v % or about 0.04 w/v %, or about 0.045 w/v % or about 0.05 w/v %, or about 0.55 w/v %, or about 0.06 w/v %, or about 0.065 w/v %, or about 0.07 w/v %, or about 0.075 w/v %, or about 0.08 w/v %, or about 0.09 w/v %, or about 0.1 w/v %, or about 0.2 w/v %, or about 0.3 w/v %, or about 0.4 w/v %, or about 0.5 w/v % or about 0.6 w/v %, or about 0.7 w/v %, or about 0.8 w/v %, or about 0.9 w/v %, or about 1 w/v %, or about 1.5 w/v %, or about 2 w/v %, of a preservative, and any amount in between. In an embodiment, the preservative at any of the above amounts is phenol. For instance, the formulation includes about 0.065 w/v % of phenol.

Tonicity modifiers include excipients such as sodium chloride, mannitol, trehalose, and similar molecules to achieve isotonic pharmaceutical injections.

Vasodilators may be used, for instance, in insulin formulations in the presence of DKP, particularly FDKP. Such vasodilators include a vasodilatory agent that can act by mediating hyperpolarization by blocking calcium ion channels, a cAMP-mediated vasodilatory agent, a cGMP-mediated vasodilatory agent or any combination thereof. In an embodiment, the vasodilatory agent that can act by mediating hyperpolarization by blocking calcium ion channels is, for example, adenosine, endothelium-derived hyperpolarizing factor, a phosphodiesterase type 5 (PDES) inhibitor, a potassium channel opener or any combination thereof. The vasodilatory agent that can act by mediating hyperpolarization by blocking calcium ion channels is adenosine. In an embodiment, the cAMP-mediated vasodilatory agent includes prostacyclin, forskolin or any combination thereof. In an embodiment, the cGMP-mediated vasodilatory agent includes nitroglycerin, a nitric oxide forming agent, amyl nitrite, nitroprusside or any combination thereof. Another vasodilatory agent that be used is treprostinil.

The vasodilatory agent is may be present in an amount of about 0.1 to about 100 mg/ml, or about 0.1 mg/ml to about 50 mg/ml, or about 0.5 mg/ml to about 25 mg/ml, or about 1 mg/ml to about 10 mg/mL. In an embodiment, the vasodilatory agent is may be present in an amount of about 0.1 mg/ml, or about 0.5 mg/ml, or about 1 mg/ml, or about 5 mg/ml, or about 10 mg/ml, or about 15 mg/ml, or about 20 mg/ml or about 25 mg/ml, or about 35 mg/ml, or about 50 mg/ml, or about 60 mg/ml, or about 75 mg/ml, or about 85 mg/ml, or about 100 mg/ml, or any amount therebetween.

Sugars and dextran molecules may be used as excipients in the formulation which are meant to associate with the insulin and DKP, and FDKP in particular, to stabilize the complex and enhance absorption. Such sugars are, for example, monosaccharides, disaccharides, and dextrans composed of mono- and di-saccharides, with functional groups such as sulfates, carboxylates, and amines, such as heparin derivatives based on dextrans that are designed with specific stabilizing properties.

The sugars and dextran molecules may be present in an amount of about 0.1 to about 100 mg/ml, or about 0.1 mg/ml to about 50 mg/ml, or about 0.5 mg/ml to about 25 mg/ml, or about 1 mg/ml to about 10 mg/mL. In an embodiment, the sugars and dextran molecules may be present in an amount of about 0.1 mg/ml, or about 0.5 mg/ml, or about 1 mg/ml, or about 5 mg/ml, or about 10 mg/ml, or about 15 mg/ml, or about 20 mg/ml or about 25 mg/ml, or about 35 mg/ml, or about 50 mg/ml, or about 60 mg/ml, or about 75 mg/ml, or about 85 mg/ml, or about 100 mg/ml, or any amount therebetween.

As used herein, aqueous solution or aqueous solvate means the solvent is greater than 50 v/v %, or greater than 55 v/v %, or greater than 60 v/v %, or greater than 65 v/v %, or greater than 70 v/v % or greater than 75 v/v % water, adjusted to be isotonic to human blood, typically with the addition of sodium chloride (NaCl) or other tonicity modifying agent. Pharmaceutically acceptable solvent or solvate means that the solvent or solvate is considered acceptable for subcutaneous injection in humans, e.g., sterile water, or water solubilizing excipients such as co-solvents in the class of polar protic and aprotic solvents such as the solubilizing excipients disclosed herein used at levels acceptable for these excipients in injectable products.

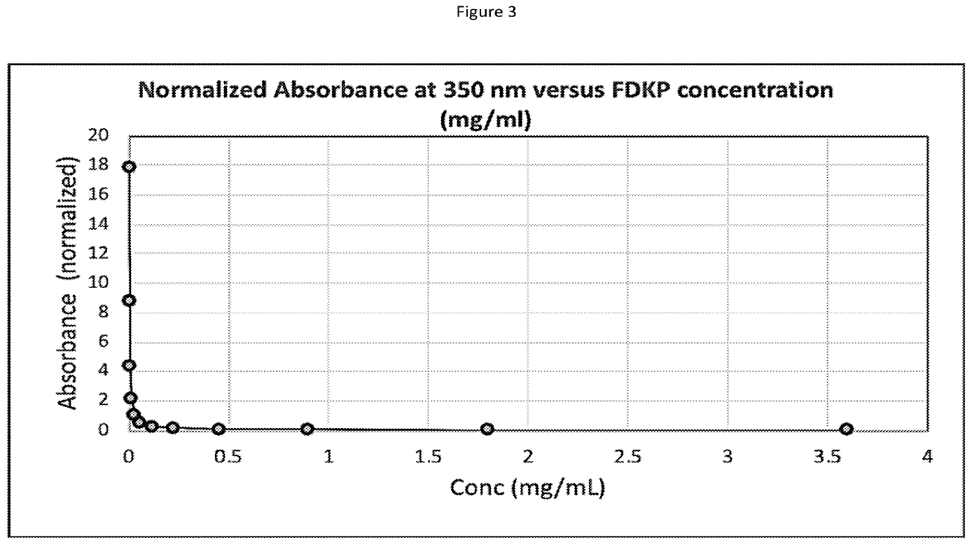

Without being restricted to any one mechanism for creating the ultra-fast insulin formulations, a diketopiperazine, for example FDKP, has effects as an absorption enhancer and permeation enhancer, a metal chelator, a surfactant, a bolaamphiphile, and an aggregation inhibitor, and promotes enhanced absorption through a combination of these effects, including enhancing the amount of monomer insulin in solution upon injection. For example, the fumaric acid charges on the FDKP may serve to charge-mask insulin positive charges, reducing the overall charge on the insulin, and making it more likely to be absorbed, especially when formulations have a pH of about 6.0 to about 7.2, as described herein. Additionally, with regard to solubility, it has been found that a diketopiperazine, for example FDKP, forms micelles at a concentration of 0.03 mg/ml (0.062 mM) or more, as shown in FIG. 3.

In addition, the combination of excipients used in the formulation have similar and additive effects for enhancing absorption, as well as enhancing stability of insulin solutions. Likewise, without being restricted to any one mechanism which can be used for creating the stabilized glucagon formulations, FDKP has effects as a metal chelator, a surfactant, a bolaamphiphile, and an aggregation inhibitor. In addition, the combination of excipients used in the formulation have similar and additive effects for enhancing stability of glucagon solutions.

In an embodiment, the ultra fast-acting subcutaneously injectable formulation is a solvated complex of a diketopiperazine, preferably FDKP, and monomeric insulin, in various aqueous solutions, including water, 10 mM phosphate, 10 mM phosphate buffered saline, 10 mM tris, 10 mM arginine either alone or in any combination. In an embodiment, the formulation includes about 30 mg/ml to about 40 mg/ml, for instance, about 36 mg/ml of FDKP, and about 4 mg/ml to about 4.5 mg/ml of monomeric insulin. In an embodiment, the formulation further comprises at least one additional excipient, for example a solubilizing excipient and/or a preservative. In an embodiment, the preservative is m-cresol in an amount of about 2 mg/ml to about 4 mg/ml, for instance 3 mg/ml. In an embodiment, the solubilizing excipient is NMP, and when NMP is used in the formulation, it may be present in an amount of about 10 wt % to about 15 wt % of the total formulation. The pH of the formulation is about 6.0 to about 7.2, or about 6.0 to about 6.9, or about 6.0 to about 6.8, or about 6.4 to about 6.8.

In an embodiment, the ultra fast-acting subcutaneously injectable formulation comprises insulin, such as a monomeric insulin, in various aqueous solutions including water, 10 mM phosphate, 10 mM phosphate buffered saline, 10 mM tris, 10 mM arginine either alone or in any combination, with one or more excipients, excluding a diketopiperazine. In an embodiment, the formulation includes about 4 mg/ml to about 4.5 mg/ml of monomeric insulin. In an embodiment, the one or more excipients for example a solubilizing excipient, for example, NMP in the ranges above, and the preservative m-cresol in an amount of about 2 mg/ml to about 4 mg/ml, for instance about 3 mg/ml. The pH of the formulation is about 6.0 to about 7.2, or about 6.0 to about 6.9, or about 6.0 to about 6.8, or about 6.4 to about 6.8.

In an embodiment, the stabilized subcutaneously injectable glucagon formulation is a solvated complex of a diketopiperazine, preferably FDKP, and glucagon, in various aqueous solutions, including water, 10 mM phosphate, 10 mM phosphate buffered saline, 10 mM tris, 10 mM arginine either alone or in any combination. In an embodiment, the formulation includes about 5 mg/ml to about 10 mg/ml of FDKP and about 0.3 mg/ml to about 0.6 mg/ml of glucagon. In an embodiment, the formulation further comprises at least one additional excipient, for example a solubilizing excipient and/or a preservative. In an embodiment, the preservative is phenol in an amount of about 0.02 wt % to about 1 wt %, for instance about 0.065 wt %. In an embodiment, the formulation includes about 0.05 wt % to about 0.5 wt %, for instance about 0.1 wt % of EDTA. In an embodiment, the formulation includes about 5 wt % to about 15 wt %, for instance about 10 wt % of beta cyclodextrin. In an embodiment, the solubilizing excipient is NMP, and when NMP is used in the formulation, it may be present in an amount of about 5 wt % to about 25 wt %, or about 10 wt % to about 25 wt % or about 15 wt % to about 25 wt %, or about 10 wt % to about 15 wt % of the total formulation. The pH of the formulation is about 6.4 to about 6.8, or about 7.0 to about 7.9, or about 7.3 to about 7.9, or about 7.6 to about 7.9.

In an embodiment, the stabilized subcutaneously injectable glucagon formulation in various aqueous solutions including water, 10 mM phosphate, 10 mM phosphate buffered saline, 10 mM tris, 10 mM arginine either alone or in any combination, with one or more excipients, excluding a diketopiperazine. In an embodiment, the preservative is phenol in an amount of about 0.02 wt % to about 1 wt %, for instance 0.065 wt %. In an embodiment, the formulation includes about 0.05 wt % to about 0.5 wt %, for instance about 0.1 wt % of EDTA. In an embodiment, the formulation includes about 5 wt % to about 15 wt %, for instance 10% of beta cyclodextrin. In an embodiment, the solubilizing excipient is NMP, and when NMP is used in the formulation, it may be present in an amount of about 5 wt % to about 25 wt %, or about 10 wt % to about 25 wt % or about 15 wt % to about 25 wt %, or about 10 wt % to about 15 wt % of the total formulation. The pH of the formulation is about 6.4 to about 6.8, or about 7.0 to about 7.9, or about 7.3 to about 7.9, or about 7.6 to about 7.9.

It also has been found that the ultra fast-acting insulin formulations as disclosed herein are stable against aggregation, fibrillation and precipitation. Moreover, such stability may be attained even if the pH is up to about 7.9. This is particularly notable, because stability may be a consideration in warmer climates. Thus, the present invention also provides stabilized (stable) subcutaneously injectable formulations comprising insulin, with or without a diketopiperazine, such as FDKP, in an aqueous solution, where the pH is about 6.0 to about 7.9. In an embodiment, the pH of a stabilized (stable) insulin formulation may be about 7.3 to about 7.9. In an embodiment, a stabilized (stable) insulin formulation may include an excipient such as NMP at the contents described above. If needed, the pH of these stabilized formulations may adjusted to provide ultra fast-acting subcutaneously injectable formulations.

To prepare the ultra fast-acting insulin solutions of this invention, one or more diketopiperazines as described herein, e.g., having an acidic side chain, are mixed with insulin in an aqueous solution having a pH in which the insulin is stable, generally a pH of about 7.4 or lower, or about 7.3 or lower, or about 7.2 or lower, or about 7.1 or lower, preferably at a pH of about 7. The amount of insulin to diketopiperazine is from about 0.1 wt % to about 50 wt %, or about 0.1 wt % to about 45 wt %, or about 0.1 wt % to about 40 wt %, or about 0.1 wt % to about 35 wt %, or about 0.1 wt % to about 30 wt %, or about 0.1 wt % to about 25 wt %, or about 0.1 wt % to about 20 wt %, or about 0.1 wt % to about 15 wt %, or about 0.1 wt % to about 10 wt %, or about 0.1 wt % to about 5 wt %, or about 0.5 wt % to about 50 wt %, or about 0.5 wt % to about 45 wt %, or about 0.5 wt % to about 40 wt %, or about 0.5 wt % to about 35 wt %, or about 0.5 wt % to about 30 wt %, or about 0.5 wt % to about 25 wt %, or about 0.5 wt % to about 20 wt %, or about 0.5 wt % to about 15 wt %, or about 0.5 wt % to about 10 wt %, or about 0.5 wt % to about 5 wt %, from about 1 wt % to about 50 wt %, or about 1 wt % to about 45 wt %, or about 1 wt % to about 40 wt %, or about 1 wt % to about 35 wt %, or about 1 wt % to about 30 wt %, or about 1 wt % to about 25 wt %, or about 1 wt % to about 20 wt %, or about 1 wt % to about 15 wt %, or about 1 wt % to about 10 wt %, or about 1 wt % to about 5 wt %. The order of addition of the diketopiperazine and insulin into the aqueous solution is not significant. The mixture of diketopiperazine and insulin in the aqueous solution is then acidified to a pH between about 4.5 to about 5.9, or about 5.0 to about 5.8 to ensure that any hexameric insulin zinc complex or insulin dimers disassociate and monomeric insulin complexes with the one or more diketopiperazines and forms particles in the solution. Such acidification may be accomplished with many common acids, although HCl is preferred. The particles of the complex of monomeric insulin and diketopiperazine are then separated from the aqueous solution so as to remove zinc that is present in the supernatant. A person of ordinary skill will understand that there are many ways to remove the supernatant after particle precipitation, such as centrifugation or filtration. Preferably, the suspension resulting after particle precipitation is spun down and the supernatant is pulled off. Optionally, the resulting precipitate can be washed with an acidified aqueous solution having a pH of about 4.5 to about 5.9, or about 5.0 to about 5.8 to further purify the insulin. Once the supernatant has been removed and any optional washing completed, the resulting precipitate may either (i) be placed in an acidified pharmaceutically acceptable aqueous solvate and the pH raised to between about 6.0 to about 6.9, preferably about 6.4 to about 6.8, to cause the monomeric insulin diketopiperazine complex to dissolve in the aqueous solvate or (ii) be added to a pharmaceutically acceptable aqueous solvate having a resultant pH between about 6.0 to about 6.9, preferably about 6.4 and 6.8 so as to provide the ultra fast-acting subcutaneously injectable insulin solution of the present invention. Typically, the resultant solution is adjusted to a range of about 25 to about 1000 International Units (IU) of insulin per ml of solution, more preferably about 100 IU/ml to about 500 IU/ml, for instance about 100 IU/ml to about 300 IU/ml or about 100 IU/ml to about 200 IU/ml.

The stabilized subcutaneously injectable glucagon solutions of the invention may be prepared in the same manner as described herein for the ultra fast-acting subcutaneously injectable insulin solutions of the invention.

In an embodiment, FDKP solutions are prepared for use in glucagon and insulin formulations. In an embodiment, the disodium salt of FDKP can be prepared by placing FDKP (1.5 gm, 3.315 mmol) into a 50 mL glass reaction flask with 10 mL of water and stirring. Sodium bicarbonate (584.8 mg, 6.96 mmol) can be added in small portions over a period of 2 hours with continuous stirring at ambient temperature. When about 90% of the bicarbonate solids have been added, the reaction can be heated to 50° C. during completion of the final addition of bicarbonate solids (about 10 minutes). Heating at 50° C. can be continued for another 10 minutes until all FDKP is in solution and clear. The solution can be frozen and lyophilized to remove the water and create solids that were readily soluble in aqueous vehicles described herein for preparing formulations described herein.

To test the pH of the Na₂-FDKP solution in water, 36 mg of the resulting solid can be dissolved in 1 mL of water to achieve a 36 mg/ml solution of FDKP. The pH of the resulting aqueous solution, may be, for instance, about pH 8.2. To adjust the pH to 7.4, 0.1N HCl (10 μL) may be added. The osmolality of the resulting pH 7.4 solution can be about 210 mOsm/kg. For the preparation of insulinactive formulations, a solution of about 30 mg/ml to about 40 mg/ml FDKP in water, for instance a 36 mg/ml solution of FDKP in water, can be prepared as described above and adjusted to the appropriate pH, as described herein, before carrying on to next steps in the formulation process including adding the insulin solution to the FDKP solution and adjusting to the final desired pH. For preparation of glucagon active formulations, a solution of about 5 mg/ml to about 10 mg/ml FDKP in water can be prepared as described above and adjusted to the appropriate pH, as described herein, before carrying on to next steps in the formulation process including adding the glucagon solution to the FDKP solution and adjusting to the final desired pH.

The ultra fast-acting subcutaneously injectable insulin solution and/or the stabilized subcutaneously injectable insulin solution of the invention may be advantageously used in an injection system for patients in need of ultra fast-acting insulin and/or stabilized insulin. Such injection systems will include a reservoir for holding a solution of the invention, wherein the reservoir communicates with one or more needles for subcutaneous invention. As used herein, reservoir means a holding place for the solution which may be, for example, a liquid container or a polymer matrix. The reservoir must communicate with one or more needles for subcutaneous injection into a patient using such a system. The injection systems of the invention include, for example, pumps, pens, and patch pumps, and microneedle pumps. Such pumps, pens, patches, and microneedle pumps are well known in the art. Examples include, without limitation, the MINIMED™ 670G Insulin Pump System manufactured by Medtronic; the OMNIPOD® Insulin management System manufactured by Insulet Corporation; or Tandem T:SLIM X2™ Insulin Pump manufactured by Tandem Diabetes Care, Inc. It is also well understood by those skilled in the art that many types of needles and soft catheters exist for use in the subcutaneous injection systems of the invention.

A particularly preferred injection system of the present invention is a bimodal injection system that is comprised of a first reservoir of the ultra fast-acting subcutaneously injectable insulin solution of the invention and a second reservoir of the stabilized subcutaneously injectable glucagon solution of the invention. The first and second reservoirs, communicate with one or more needles for subcutaneous injection. Each reservoir may communicate with the same one or more needles for subcutaneous injection or each reservoir may communicate with an independent one or more needles for subcutaneous injection. The bimodal injection system will also include means for introducing a therapeutically effective amount of the ultra fast-acting insulin solution of the invention when a hyperglycemic condition is detected in the patient using the bimodal system and introducing a therapeutically effective amount of the stabilized glycogen solution when a hypoglycemic condition is detected in the patient using the bimodal system.

Persons of ordinary skill will understand that Continuous Glucose Monitoring (CGM) devices are now available and are either separate or part of a “Artificial Pancreas” system. The CGM is coupled to computer algorithms (often blue-toothed to a patient's smart phone) that “learn” how the particular patient is likely to respond to an insulin injection and makes a suggestion to the patient to allow the pump to administer the recommended dose.

Systems for detecting blood glucose levels and delivering insulin are known. Examples include, U.S. Patent Application Publication No. 2019/0015515, U.S. Pat. Nos. 10,279,106, 10,350,354, 10,188,325, 9,452,259, all of which are incorporated by reference in their entirety.

It should be appreciated that the bimodal injection system of the invention improves on such disclosed systems by providing a highly efficient way to maintain a patient's blood sugar level in a healthy range when employing both the ultra fast-acting subcutaneously injectable insulin solution and the stabilized subcutaneously injectable glucagon solution described herein.

In another embodiment, the invention is directed to pen and pump devices with monitors for dose number and dose quantity to increase patient compliance, and for connection of these analytical measures to smart phone, tablet, or electronic records systems based on applications for patient and physician monitoring of therapy.

In another embodiment, the injection needle has a glucose sensor affixed to it such that an accurate blood glucose value is recorded when the insulin or glucagon is injected. This value is also tracked wirelessly on the device and the data wirelessly uploaded to an application for review on a smart phone, tablet, or in patient electronic medical record systems.

In another embodiment, a port system for subcutaneous injection is used subcutaneously and is placed in the skin for up to 7 days to prevent multiple injections into the skin. Further, the port has a catheter that is embedded into the subcutaneous tissue and this catheter contains a glucose sensor wirelessly attached to the pen and attached to an application for patient and physician monitoring.

The invention also includes a method of treating a patient in need of insulin comprising the step of subcutaneously administering to the patient in need thereof a therapeutically effective amount of the ultra fast-acting subcutaneously injectable insulin solution of the invention. Patients in need of such treatment include those having diabetes mellitus, including Type 2 diabetes, Type 1 diabetes and gestational diabetes. In another embodiment, the invention includes a method of treating a patient in need of glucagon comprising the step of subcutaneously administering to the patient in need thereof a therapeutically effective amount of the stabilized subcutaneously injectable glucagon solution of the invention. Patients in need of such treatment include those suffering from hypoglycemia, persistent hyper insulinemic hypoglycemia of infancy and other hyper insulinemic conditions.

Another embodiment of the invention is directed to maintaining a healthy blood glucose level in a patient in need of such maintenance by the steps of: (i) subcutaneously administering the ultra fast-acting subcutaneously injectable insulin solution of the invention when a hyperglycemic condition is detected in the patient and (ii) subcutaneously administering the stabilized subcutaneously injectable glucagon solution of the invention when a hypoglycemic condition is detected in the patient. This method of maintaining a healthy blood glucose level can be achieved when the insulin solution and the glucagon solution are included in the same pump, although it is also envisioned for the patient to use one patch or pump containing the ultra fast-acting insulin solution of the invention while simultaneously using a second patch or pump containing the stabilized glucagon solution of the invention.

Formulations described herein can be characterized after preparation and on stability and purity using the following experimental methods. Reversed phase HPLC was used to determine the concentration of insulin and glucagon in formulations using a standard curve of each. The same reversed phase HPLC method was used to determine the purity of the insulin and glucagon in formulations by comparing the integrated peak area of the main peak of insulin or glucagon versus the total area of all impurities in the HPLC chromatogram. Typically, it is desired to have greater than 95%, or greater than 96%, or greater than 97%, or greater than 98% purity of the insulin or glucagon as the main peak when the formulations are prepared. Stability samples with lower than 90% purity are considered to be inadequate. For glucagon, stability can be accessed based on a 7 day period in a rotating incubator at 37° C. For insulin, stability can be assessed based on sample storage in a refrigerator for 2 year at 2° C. to 8° C., or 12 months at 25° C., or 6 months at 40° C. Stability samples are considered to be inadequate that decrease by more than 10% in concentration or 10% in purity by HPLC methods, and show more than 5% of high molecular weight aggregates by HPLC with SEC. In addition, HPLC size exclusion (SEC) columns were used to determine the amount of high molecular weight insulin or glucagon in the samples, which is a sign of aggregation of the insulin or glucagon. Upon preparation of formulations, high molecular weight content is typically less than 1 wt %, and samples on stability should typically have less than 3 wt % to 5 wt % high molecular weight content to be considered adequate. Physical methods may be used to characterize the formulations such as visual clarity and lack of particulate matter. If a solution has lost clarity or become hazy, or has visible particles floating in it, it is considered inadequate. In addition, pH can be determined of each formulation on preparation and on stability using a standard pH meter. Finally, osmolality of formulations can be determined using a standard osmometer.

### EXAMPLES

### Example 1

Regular recombinant human insulin was put into a water solution with a diketopiperazine (DKP), specifically 2,5-diketo-3,6-di(4-fumarylaminobutyl)piperazine (FDKP) at a pH of 7.4. The solution pH was then lowered to a pH of about 5 by the addition of aqueous HCL. This resulted in the formation of particles, which when dried had a mean diameter of 2 microns. The supernatant in which the particles were suspended contained the zinc from the hexameric insulin. The particles were centrifuged and washed with water at a pH of about 5 to facilitate removal of the zinc. After washing, the particles were suspended in a reverse osmosis (RO) water solution that was adjusted to a pH between 6.4 and 6.8. The resulting solution was clear by visual inspection. Dynamic Laser Light Scattering (DLS) experiments at this pH range showed the clear solution contained a complex significantly greater in size than hexameric insulin, indicating an association of the insulin molecules with the DKP. In addition, as the zinc has been removed or significantly reduced, there is a reduced possibility of hexamer formation by the monomeric insulin associated with the DKP in the solution.

### Example 2

Glucagon was put into a water solution with FDKP at a pH of 7.4. The solution pH was then lowered to a pH of about 5 by the addition of aqueous HCL. This resulted in the formation of particles, which when dried had a mean diameter of 2 microns. (or about 8 microns for the Succinic acid DKP). The particles were centrifuged and washed with water at a pH of about 5 to assist in purification. After washing, the particles were suspended in a reverse osmosis (RO) water solution that was adjusted to a pH between 6.4 and 6.8. The resulting solution was clear by visual inspection. Dynamic Laser Light Scattering (DLS) experiments at this pH range showed the clear solution contained a complex significantly greater in size than glucagon itself, indicating an association of the glucagon molecules with the DKP. The resulting solution was stable and did not exhibit gelling under visual examination. The formulation was stable, e.g., lost less than 5% of activity after two weeks storage as a liquid at 30° C.

### Example 3

Recombinant human insulin (RHI) was prepared in aqueous solutions containing Na₂-FDKP as shown in Table 1.

TABLE 1

| | RHI | Na₂FDKP | m-cresol | Buffer | |
|---|---|---|---|---|---|
| Formulation | (mg/ml) | (mg/ml) | (mg/ml) | (10 mM) | pH |
| A | 4.16 | 36 | 3.0 | Phosphate | 7.0 |
| B | 4.43 | 36 | 3.0 | Phosphate | 7.4 |
| C | 4.1 | 36 | 3.0 | L-Arginine | 7.2 |
| D | 4.0 | 36 | 3.0 | Tris | 7.2 |
| E | 4.2 | 36 | 3.0 | Water | 7.0 |

For the formulations in Table 1, 20 ml of 10 mM buffer was made for the 5 different formulations. To each buffer, m-cresol was added at a concentration of 3 mg/ml. 75 mg of FDKP was weighed into 5 different 5 ml Eppendorf tubes, one for each buffer. To each tube, 1.5 ml of buffer was added and inverted to mix. A standard solution of insulin was made by weighing 100 mg of insulin into a 5 ml Eppendorf and adding 2 ml of 0.1N HCL. To each 5 ml centrifuge tube containing FDKP, buffer, and m-cresol, 160 μl of insulin solution was added. The pH was adjusted to values in the above table and then volume was brought up to 2 ml. The solutions were filtered through an 0.2 um syringe tip filter and split into two batches for stability evaluation, one for 40° C. and the other for 25° C.

In order to determine insulin concentration and purity, a specific reversed-phase HPLC (“RP-HPLC”) method was used. The RP-HPLC method uses a Waters Quaternary pump and DAD detector using a xBridge peptide BEH C18 column, 130A, 13.5 um, 4.6 mm×150 mm with Guard Cartridge C18 4×3 mm with a column temperature of 40° C. The flow rate was 0.5 ml/min, with an injection volume of 10 ul, and UV detection of 274 nm. The insulin retention time was 13.98 minutes. Additionally, a specific reversed-phase HPLC size exclusion (“HPLC-SEC”) method was used to determine whether there are insulin aggregates or higher order complexes present in samples. The HPLC-SEC method uses an Agilent 1100 with Quaternary pump and DAD detector using a Superdeck HR THF 7.8×300 mm column, with a column temperature of 25° C. The flow rate was 0.5 ml/min, with an injection volume of 30 ul, and UV detection of 276 nm. The insulin retention time was 17.78 minutes. In this HPLC-SEC method, insulin higher molecular weights species elute as multimers at a retention time of 13 to 17 minutes, insulin in the dimer form elutes at a retention time of 17.5 minutes, and insulin in the monomeric form elutes at a retention time between 18 and 22 minutes.

Initial analysis was completed by RP-HPLC to establish the insulin concentration and purity, and size exclusion chromatography to show that there were no higher molecular weight or aggregates present. Visual observations were taken each day for a week showing that the solutions were clear and showed no precipitation or visible particulates at either temperature.

### Example 4

Recombinant human insulin was prepared in aqueous solutions containing FDKP 36 mg/ml with insulin at 4 mg/ml, and various buffers (water, 10 mM phosphate, 10 mM Tris, 10 mM arginine) including 3 mg/ml m-cresol in all formulations with pH ranging from pH 7.0 to 7.9.

Stability and purity were measured in accordance with the methods described in Example 3. Initial analysis was completed by RP-HPLC to establish the insulin concentration and purity, and size exclusion chromatography to show that there were no higher molecular weight or aggregates present. Visual observations were taken each day for a week showing that the solutions were clear and showed no precipitation or visible particulates at either temperature. There was no change in any parameter on stability at 25° C. and 40° C., as shown in Table 2 (marked as “✓”).

TABLE 2

| | 4 mg/ml RHI | | | | | Purity | |
|---|---|---|---|---|---|---|---|
| | 36 mg/ml FDKP | | | | | (RP- | Aggregation |
| | 3 mg/ml m-cresol | Temp | Visual | pH | Osmolality | HPLC) | (SEC) |
| 1 | Water pH 7.4 | 25° C. | Clear | 7.6 | 223 | ✓ | ✓ |
| | Water pH 7.0 | 40° C. | Clear | 7.4 | 226 | ✓ | ✓ |
| 2 | 10 mM Phos 7.0 | 25° C. | Clear | 7.6 | 255 | ✓ | ✓ |
| | 10 mM Phos 7.0 | 40° C. | Clear | 7.5 | 247 | ✓ | ✓ |
| 3 | 10 mM Phos 7.4 | 25° C. | Clear | 7.5 | 245 | ✓ | ✓ |
| | 10 mM Phos 7.4 | 40° C. | Clear | 7.6 | 241 | ✓ | ✓ |
| 4 | 10 mM Tris pH 7.3 | 25° C. | Clear | 7.9 | 253 | ✓ | ✓ |
| | 10 mM Tris pH 7.3 | 40° C. | Clear | 7.7 | 280 | ✓ | ✓ |
| 5 | 10 mM Arg pH 7.3 | 25° C. | Clear | 7.4 | 264 | ✓ | ✓ |
| | 10 mM Arg pH 7.3 | 40° C. | Clear | 7.4 | 254 | ✓ | ✓ |

### Example 5

Recombinant human insulin was prepared in aqueous solutions with insulin at 4 mg/ml, and various buffers (water, 10 mM phosphate, 10 mM Tris, 10 mM arginine) including 3 mg/ml m-cresol in all formulations with pH ranging from pH 7.0 to 7.9. No DKP is in the formulations.

Stability and purity were measured in accordance with the methods described in Example 3. Initial analysis was completed by RP-HPLC to establish the insulin concentration and purity, and size exclusion chromatography to show that there were no higher molecular weight or aggregates present. Visual observations were taken each day for a week showing that the solutions were clear and showed no precipitation or visible particulates at either temperature. There was no change in any parameter on stability at 25° C. and 40° C., as shown in Table 3 (marked as “✓”).

TABLE 3

| | | | | | Purity | |
|---|---|---|---|---|---|---|
| | 4 mg/ml RHI | | | | (RP- | Aggregation |
| | 3 mg/ml m-cresol | Temp | Visual | pH | HPLC) | (SEC) |
| 1 | Water pH 7.4 | 25° C. | Clear | 7.4 | ✓ | ✓ |
| | Water pH 7.0 | 40° C. | Clear | 7.7 | ✓ | ✓ |
| 2 | 10 mM Phos 7.0 | 25° C. | Clear | 7.5 | ✓ | ✓ |
| | 10 mM Phos 7.0 | 40° C. | Clear | 7.4 | ✓ | ✓ |
| 3 | 10 mM Phos 7.4 | 25° C. | Clear | 7.7 | ✓ | ✓ |
| | 10 mM Phos 7.4 | 40° C. | Clear | 7.6 | ✓ | ✓ |
| 4 | 10 mM Tris pH 7.3 | 25° C. | Clear | 7.6 | ✓ | ✓ |
| | 10 mM Tris pH 7.3 | 40° C. | Clear | 7.7 | ✓ | ✓ |
| 5 | 10 mM Arg pH 7.3 | 25° C. | Clear | 7.3 | ✓ | ✓ |
| | 10 mM Arg pH 7.3 | 40° C. | Clear | 7.7 | ✓ | ✓ |

### Example 6

Stabilized glucagon formulations were prepared at concentrations between 0.3 and 0.6 mg/ml as measured by reversed-phase HPLC in various buffers (water, 10 mM phosphate, 10 mM Tris, 10 mM arginine) with 10% to 25% of NMP in pH ranging from 7.6 to 7.9. FDKP concentration ranged from 5 to 10 mg/ml. Phenol and benzyl alcohol preservatives were used in some samples.

In order to determine glucagon concentration and purity, a specific reversed-phase HPLC (“RP-HPLC”) method was used. The RP-HPLC was a Agilent Quaternary pump and DAD detector using a xBridge peptide BEH C18 column, 130A, 13.5 um, 4.6 mm×150 mm with Guard Cartridge C18 4×3 mm with a column temperature of 40° C. The flow rate was 1 ml/min, with an injection volume of 10 ul, and UV detection of 274 nm. The insulin retention time was 14.01 minutes. Additionally, a specific reversed-phase HPLC size exclusion (“HPLC-SEC”) method was used to determine whether there are glucagon aggregates or higher order complexes present in samples. The HPLC-SEC method uses an Agilent 1100 with Quaternary pump and DAD detector using a Superdeck HR THF 7.8×300 mm column, with a column temperature of 25° C. The flow rate was 0.5 ml/min, with an injection volume of 30 ul, and UV detection of 273 nm. The insulin retention time was 21 minutes. In this HPLC-SEC method, glucagon higher molecular weights species elute as multimers at a retention time of 18 minutes and as aggregates at a retention time of 15 minutes.

The stability of the formulations is shown in Table 4. For a number of samples, there was minimal change in the parameters upon shaking in an incubator at 37° C. for 7 days.

TABLE 4

| | % D7 | % Purity |
|---|---|---|
| | Conc/Day0 | D7 |
| 10 mM Phos with 10% NMP Control + 5 mg/ml FDKP | 96% | 99% |
| 10 mM Phos with 15% NMP + 5 mg/ml FDKP | 92% | 99% |
| 10 mM Arg with 15% NMP + 5 mg/ml FDKP | 77% | 94% |
| 10 mM Tris with 15% NMP + 5 mg/ml FDKP | 77% | 92% |
| 10 mM Phos with 15% NMP with 10% Beta Cyclodextrin + 5 mg/ml | 76% | 94% |
| FDKP | | |
| 10 mM Phos buffer with 25% NMP + 10 mg/ml FDKP | 105% | 97% |
| 10 mM Phos with 10% NMP + 0.065% Phenol + 5 mg/ml FDKP | 85% | 90% |
| 10 mM Phos with 25% NMP + 0.065% Phenol + 5 mg/ml FDKP | 92% | 98% |
| 10 mM Phos with 10% NMP + 4% Benzyl Alcohol + 5 mg/ml FDKP | 94% | 94% |
| 10 mM Phos with 10% NMP + 0.1% EDTA in Phos + 5 mg/ml | 75% | 96% |
| FDKP | | |

### Example 7

Stabilized glucagon formulations were prepared at concentrations between 0.3 and 0.6 mg/ml as measured by reversed-phase HPLC in various buffers (water, 10 mM phosphate, 10 mM Tris, 10 mM arginine) with 5% to 25% of NMP in pH ranging from 7.6 to 7.9. Phenol and EDTA and beta cyclodextrin were used in some samples. No DKP was used.

In order to determine glucagon concentration and purity, the method described in Example 6 was used. The stability of the formulations is shown in Table 5. For a number of samples, there was minimal change in the parameters upon shaking in an incubator at 37° C. for 7 days.

TABLE 5

| | % D7 | % Purity |
|---|---|---|
| | Conc/Day0 | D7 |
| 10 mM Phos with 5% NMP | 83% | 96% |
| 10 mM Phos with 10% NMP | 84% | 99% |
| 10 mM Phos with 15% NMP | 106% | 97% |
| 10 mM Arg with 15% NMP | 98% | 97% |
| 10 mM Tris with 15% NMP | 83% | 91% |
| 10 mM Phos with 15% NMP | 79% | 94% |
| with 10% Beta Cyclodextrin | | |
| water with 15% NMP with | 75% | 86% |
| 10% Beta Cyclodextrin | | |
| 10 mM Phos with 25% NMP | 112% | 98% |
| 10 mM Phos with 10% NMP + 0.065% Phenol | 105% | 96% |
| 10 mM Phos with 25% NMP + 0.065% Phenol | 108% | 96% |
| 10 mM Phos with 10% NMP + 0.1% EDTA | 57% | 99% |

### Example 8

Recombinant human insulin were prepared in ultra-fast acting subcutaneous injectable formulations in aqueous solutions containing FDKP at pH 6.5 and 7.4, as summarized in Table 6. All formulation preparation was conducted in a laminar flow hood and the final solutions were filtered through 0.2 micron filters. Both formulations were injected into the subcutaneous tissue of diabetic minipigs (Yucatan minipigs rendered diabetic by treatment with alloxan).

TABLE 6

| | RHI | Na₂FDKP | m-cresol | Buffer | | Osmolality |
|---|---|---|---|---|---|---|
| Formulation | (mg/ml) | (mg/ml) | (mg/ml) | (10 mM) | pH | (mOsm/kg) |
| CP-001-001 | 4.16 | 36 | 3.0 | phosphate | 6.5 | 280 |
| CP-002-001 | 4.43 | 36 | 3.0 | phosphate | 7.49 | 264 |

10 ml of a 10 mM phosphate buffer was made at two different pHs for the two formulations, one at pH 6.5 and another at pH 7.5. To each phosphate buffer, m-cresol was added at a concentration of 3 mg/ml. 118.5 mg of FDKP was weighed into two 5 ml Eppendorf tubes, one for each pH. 12 mg of recombinant human insulin (Aldrich) was added to each of the tubes containing dry FDKP solids. Each of the buffers was added in a quantity of 3 ml to their respective solid mixtures in the tubes and vortexed to solubilize. The resulting solutions were adjusted to pH 6.55 and pH 7.49 respectively using NaOH and HCl. The complete mixture was filtered through an 0.22 μm syringe tip filter into a 2 ml autoclaved serum vial and the batches split. 1 ml was moved to a new vial and crimped for the animal study. The remaining volume (2 mL) was used for analysis of the sample and the remainder split into 3 vials for stability evaluation at 5° C., 25° C., and 40° C. The samples were analyzed by RP-HPLC and SEC-HPLC as described in Example 3. The stability of the formulations is shown in Table 7.

TABLE 7

| | 5° C. | 5° C. | 25° C. | 25° C. | 40° C. | 40° C. |
|---|---|---|---|---|---|---|
| | Stability | Stability | Stability | Stability | Stability | Stability |
| | % T0 | % T0 | % T0 | % T0 | % T0 | % T0 |
| | Conc | Purity | Conc | Purity | Conc | Purity |
| 1 | 97% | 101% | 104% | 104% | 96% | 101% |
| 2 | 93% | 101% | 94% | 101% | 92% | 101% |

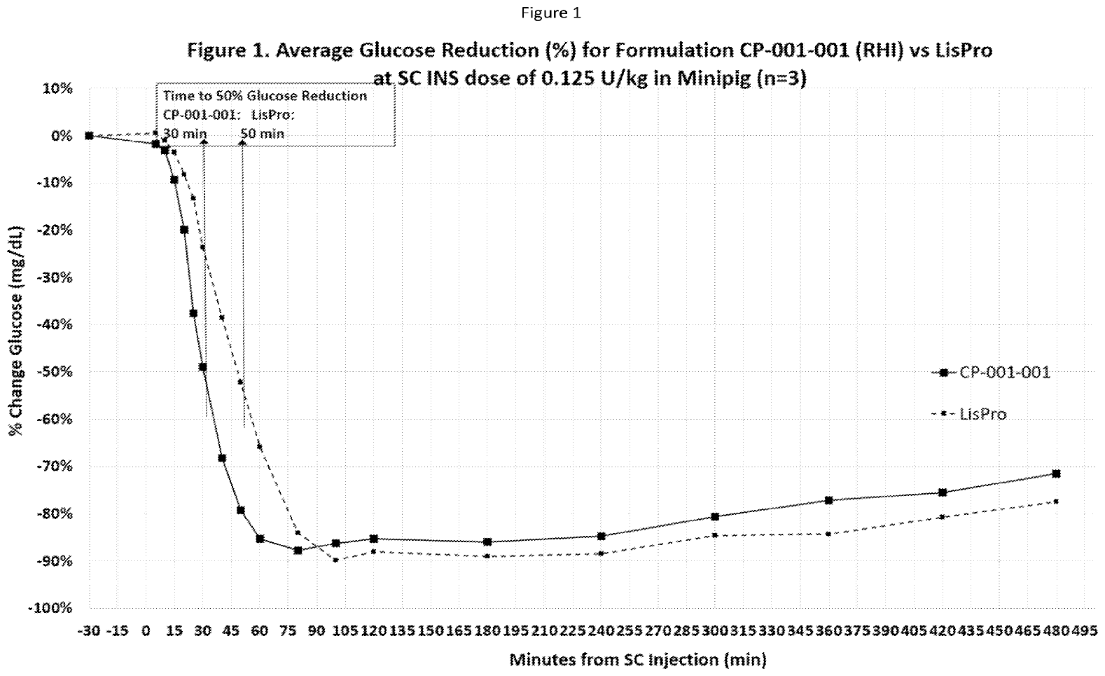

The glucose response from the pH 6.5 formulation compared to a subcutaneous injection of standard lispro insulin is shown in FIG. 1. The data clearly indicate absorption of insulin and glucose response from the ultra-rapid FDKP insulin formulation is more rapid than lispro. Time to 50% glucose reduction from the formulation is 30 minutes, while that from standard lispro is 50 minutes. The result for the ultra-fast insulin formulation is for recombinant human insulin and shows a faster absorption than recombinant human insulin as well. Based on this data set, it is expected that the absorption rate for lispro in the FDKP formulation at pH 6.5 would be several fold faster than for standard lispro, and comparable to or better than lyumjev.

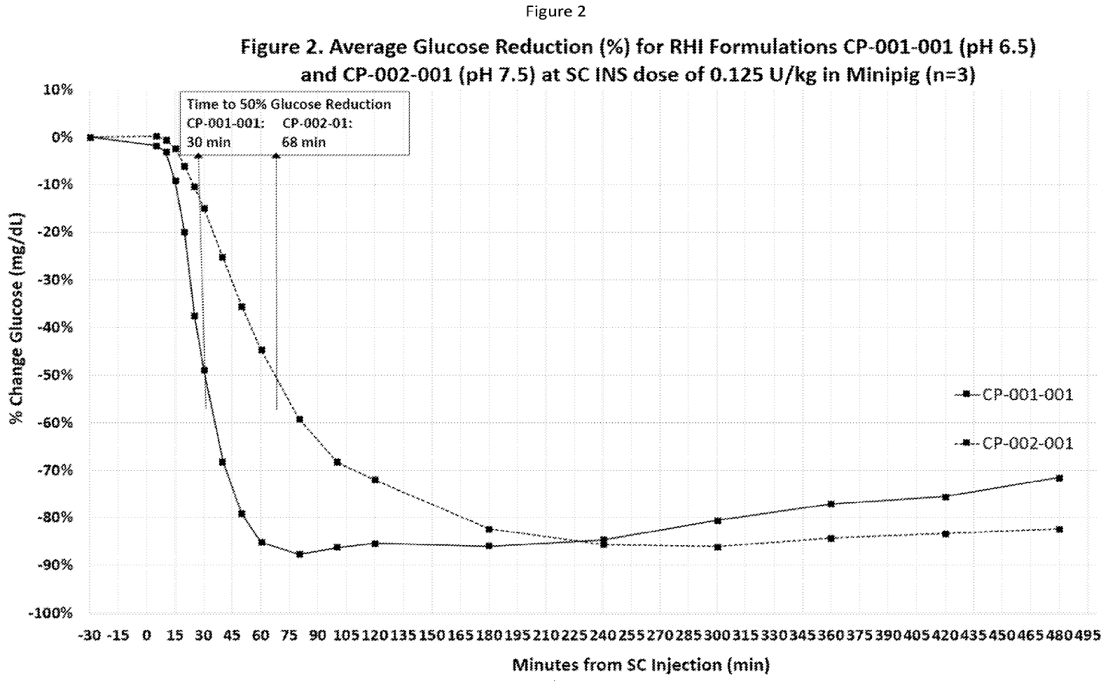

The glucose response from the pH 7.5 formulation compared to a subcutaneous injection of the ultra-fast acting pH 6.5 formulation, both containing FDKP, are shown in FIG. 2. The data indicate absorption of insulin and glucose response from the ultra-rapid FDKP insulin formulation at pH 6.5 is much more rapid than the pH 7.5 formulation containing FDKP. Time to 50% glucose reduction from the pH 6.5 FDKP formulation is 30 minutes while that from pH 7.5 FDKP formulation was 68 minutes, a value that is slower than lispro insulin absorption. The strong pH dependency of the absorption rate was not anticipated. While not wanting to be held to a specific mechanism for the rapid absorption, the lower pH solution appears to cause there to be less charge on the FDKP, which would make it more hydrophobic, and therefore more likely to bind tightly to the insulin molecule, thereby reducing the overall charge on insulin, causing there to be more monomers in solution, and enhancing the absorption by several mechanisms.

### Example 9

A standard solution of insulin (using recombinant human insulin) was made in 0.05N aqueous HCl. An aliquot (1 mg) of the solution was transferred into 4 different Eppendorf tubes and 9 mg of solid Na₂-FDKP was added into each tube. A milky suspension formed immediately. The pH was adjusted to the values between pH 6.0 and pH 7.4 using 0.1N aqueous NaOH. Samples of insulin alone were compared to FDKP alone and to the solution of insulin and FDKP. The insulin alone samples were all insoluble. The FDKP alone samples were soluble at pH 7.4, partially soluble at pH 7.0 and insoluble below pH 7.0. All samples containing 9 mg/ml FDKP and 1 mg/ml insulin were soluble at all pH values. The pH 6.0 solution of FDKP and insulin was clear at 25° C. for 2 days and slowly became turbid thereafter.

TABLE 8

| | | | | Insulin | |
|---|---|---|---|---|---|
| | | | | (1 mg/ml) | |
| | | FDKP | Insulin | plus FDKP | Notes on |
| Entry | pH | (9 mg/mL) | (1 mg/mL) | (9 mg/mL) | solution |
| 1 | 6.0 | Insoluble | insoluble | soluble | Clear for 2 day |
| 2 | 6.5 | Insoluble | Insoluble | soluble | Clear for 1 week tested |
| 3 | 7.0 | Partial | Insoluble | soluble | Clear for 1 week tested |
| 4 | 7.4 | Soluble | Insoluble | soluble | Clear for 1 week tested |

### Example 10

Recombinant human insulin was prepared in a subcutaneous injectable formulation in aqueous solutions containing FDKP 36 mg/ml with insulin at 4 mg/ml, and various buffers (water, 10 mM phosphate, 10 mM phosphate buffered saline) including 3 mg/ml m-cresol in all formulations with pH ranging from pH 6.0 to 7.4. Stability for these formulations was good in all cases except for some haziness observed in the pH 6.0 samples.

The stability of the formulations is shown in the Table 9 below. Parameters evaluated were clarity of solution, HPLC concentration and purity using a stability indicating HPLC method (RP-HPLC), and high molecule weight insulin using a size exclusion method (HPLC-SEC) as described above in Example 3.

TABLE 9

| | | pH | pH | pH | pH | pH |
|---|---|---|---|---|---|---|
| | Buffer System | 6.0 | 6.5 | 6.8 | 7.0 | 7.4 |
| 1 | Water | Slight Hazy | Clear | Clear | Clear | Clear |
| 2 | 10 mM Phosphate | Slight Hazy | Clear | Clear | Clear | Clear |
| 3 | 10 mM phosphate | Slight Hazy | Clear | Clear | Clear | Clear |
| | buffered saline | | | | | |

While the invention has been described with reference to exemplary embodiments, it is to be understood that the invention is not limited thereto.

1. An ultra fast-acting subcutaneously injectable insulin formulation comprising a solvated complex of a diketopiperazine and a monomeric insulin in an aqueous solution at a pH of about 6.0 to about 7.2.

2. The ultra fast-acting subcutaneously injectable insulin formulation of claim 1, wherein the pH is about 6.0 to about 7.0.

3. The ultra fast-acting subcutaneously injectable insulin formulation of claim 1, wherein the aqueous solution is water, optionally comprising sodium chloride.

4. An ultra fast-acting subcutaneously injectable insulin formulation comprising:
a solvated complex of a diketopiperazine and a monomeric in an aqueous solution at a pH of about 6.0 to about 7.2; and one or more additional excipients.

5. The ultra fast-acting subcutaneously injectable insulin formulation of claim 4, wherein the one or more additional excipients are selected from the group consisting of a buffer excipient, a solubilizing excipient, an aggregation preventer, a surfactant, an absorption enhancer, a permeation enhancer, a metal chelator, a preservative, a tonicity modifier and any combination thereof.

6. The ultra fast-acting subcutaneously injectable insulin formulation of claim 5, wherein the buffer excipient is selected from the group consisting of tris(hydroxymethyl)aminomethane, phosphate, phosphate buffered saline, arginine, glycine, phosphate-citrate, histidine, and any combination thereof.

7. The ultra fast-acting subcutaneously injectable insulin formulation of claim 5, wherein the buffer excipient is a phosphate buffer.

8. The ultra fast-acting subcutaneously injectable insulin formulation of claim 5, wherein the preservative in selected from the group consisting of such as phenol, m-cresol, benzyl alcohol, parabens and parabens esters, phenoxyethanol, benzalkonium chloride, and any combination thereof.

9. The ultra fast-acting subcutaneously injectable insulin formulation of claim 5, wherein the preservative is m-cresol, and is present in the formulation in an amount of about 1 mg/ml to about 10 mg/ml.

10. The ultra fast-acting subcutaneously injectable insulin formulation of claim 5, wherein the preservative is m-cresol.

11. The ultra fast-acting subcutaneously injectable insulin formulation of claim 5, wherein the solubilizing excipient is selected from the group consisting of dimethyl sulfoxide, N-methyl-2-pyrrolidone, ethanol, propylene glycol, glycerol, polyethylene glycol and any combinations thereof.

12. The ultra fast-acting subcutaneously injectable insulin formulation of claim 5, wherein the solubilizing excipient is N-methyl-2-pyrrolidonem and is present in the formulation in an amount of about 1 wt % to about 25 wt.

13. The ultra fast-acting subcutaneously injectable insulin formulation of claim 5, wherein the absorption enhancer and the permeation enhancer are selected from the group consisting of polysorbates, glycocolates, glycholic acid, citric acid, ethylenediaminetetraacetic acid, methyl beta cyclodextrin, beta cyclodextrin, dipalmitoylphosphatidylcholine, polyamidoamine dendrimers, and any combination thereof.

14. The ultra fast-acting subcutaneously injectable insulin solution of claim 4, wherein the aqueous solution is at a pH of about 6.0 to about 7.0.

15. The ultra fast-acting subcutaneously injectable insulin formulation of claim 1, wherein the diketopiperazine is represented by formula (I): 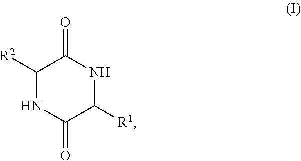
wherein R₁ and R₂ are independently X—Y—Z—W, and wherein X is a C1-C20 straight or branched or cyclic alkyl, aralkyl, alkaryl, alkenyl, alkynyl, heteroalkyl, heterocyclic, alkyl-heterocyclic or heterocyclic-alkyl;
wherein Y may be present or absent and when present is —C(O)O, —OC(O), C(O)NH, —NH, —NX, —OXO, —O, —NHC(O), —OP(O), —P(O)—, —OP(O)₂, —P(O)₂O, —OS(O)₂, or —S(O)₃;
wherein Z may be present or absent and when present is a C1-C20 straight or branched or cyclic alkyl, aralkyl, alkaryl, alkenyl, alkynyl, heteroalkyl, heterocyclic, alkyl-heterocyclic or heterocyclic-alkyl; and
wherein W is an acid group.

16. The ultra fast-acting subcutaneously injectable insulin formulation of claim 15, wherein the acid group is selected from the group consisting of cis —CH═CH—CO₂H, trans —CH═CH—CO₂H, —CH(CH₃)═CH(CH₃)—CO₂H, —(CH₂)₃—CO₂H, —CH₂CH(CH₃)—CO₂H, —CH(CH₂CO₂H)═CH₂, -(tetrafluoro)benzoic acid, -benzoic acid and —CH(NHC(O)CF₃)—CH₂—CO₂H.

17. The ultra fast-acting subcutaneously injectable insulin formulation of claim 15, wherein the diketopiperazine is selected from the group consisting of 2,5-diketo-3,6-di(4-succinylaminobutyl)piperazine, 2,5-diketo-3,6-di(4-fumarylaminobutyl)piperazine, 2,5-diketo-3,6-di(4-maleylaminobutyl)piperazine and 2,5-diketo-3,6-di(4-glutarylaminobutyl)piperazine.

18. An ultra fast-acting subcutaneously injectable insulin formulation comprising a monomeric in an aqueous solution with one or more excipients, excluding a diketopiperazine, at a pH of about 6.0 to about 7.2.

19. The ultra fast-acting subcutaneously injectable insulin formulation of claim 18, wherein the one or more excipients are selected from the group consisting of a buffer excipient, a solubilizing excipient, an aggregation preventer, a surfactant, an absorption enhancer, a permeation enhancer, a metal chelator, a preservative, a tonicity modifier and any combination thereof.

20. The ultra fast-acting subcutaneously injectable insulin formulation of claim 19, wherein the buffer excipient is selected from the group consisting of tris(hydroxymethyl)aminomethane, phosphate, phosphate buffered saline, arginine, glycine, phosphate-citrate, histidine, and any combination thereof.

21. The ultra fast-acting subcutaneously injectable insulin formulation of claim 19, wherein the buffer excipient is a phosphate buffer.

22. The ultra fast-acting subcutaneously injectable insulin formulation of claim 19, wherein the preservative in selected from the group consisting of such as phenol, m-cresol, benzyl alcohol, parabens and parabens esters, phenoxyethanol, benzalkonium chloride, and any combination thereof.

23. The ultra fast-acting subcutaneously injectable insulin formulation of claim 19, wherein the preservative is m-cresol, and is present in the formulation in an amount of about 1 mg/ml to about 10 mg/ml.

24. The ultra fast-acting subcutaneously injectable insulin formulation of claim 19, wherein the preservative is m-cresol.

25. The ultra fast-acting subcutaneously injectable insulin formulation of claim 19, wherein the solubilizing excipient is selected from the group consisting of dimethyl sulfoxide, N-methyl-2-pyrrolidone, ethanol, propylene glycol, glycerol, polyethylene glycol and any combination thereof.

26. The ultra fast-acting subcutaneously injectable insulin formulation of claim 19, wherein the solubilizing excipient is N-methyl-2-pyrrolidonem and is present in the formulation in an amount of about 1 wt % to about 25 wt %.

27. The ultra fast-acting subcutaneously injectable insulin formulation of claim 19, wherein the absorption enhancer and the permeation enhancer are selected from the group consisting of polysorbates, glycocolates, glycholic acid, citric acid, ethylenediaminetetraacetic acid, methyl beta cyclodextrin, beta cyclodextrin, dipalmitoylphosphatidylcholine, polyamidoamine dendrimers, and any combination thereof.

28. The ultra fast-acting subcutaneously injectable insulin formulation of claim 18, wherein the aqueous solution is at a pH of about 6.0 to about 7.4.

29. A stabilized subcutaneously injectable glucagon formulation comprising a solvated complex of a diketopiperazine and glucagon in an aqueous solution at a pH of about 6.4 to about 7.9.

30-47. (canceled)

48. A stabilized subcutaneously injectable glucagon formulation comprising glucagon in an aqueous solution with one or more excipients, excluding a diketopiperazine, at a pH of about 6.4 to about 7.9.

49-59. (canceled)

60. An ultra fast-acting subcutaneously injectable insulin formulation comprising a solvated complex of a diketopiperazine and a monomeric insulin, wherein the diketopiperazine is 2,5-diketo-3,6-di(4-fumarylaminobutyl)piperazine.

61. The ultra fast-acting subcutaneously injectable insulin formulation of claim 60, further comprising a buffer excipient selected from the group consisting of phosphate, phosphate buffered saline, tris(hydroxymethyl)aminomethane, arginine, and any combination thereof, and a preservative,
wherein the formulation has a pH of about 6.0 to about 7.2.

62. An ultra fast-acting subcutaneously injectable insulin formulation comprising a monomeric insulin, a buffer excipient selected from the group consisting of phosphate, phosphate buffered saline, tris(hydroxymethyl)aminomethane, arginine, and any combination thereof, and a preservative, wherein the formulation has a pH of about 6.0 to about 7.2.

63-64. (canceled)

65. An injection system for subcutaneous injection of insulin comprising a reservoir communicating with one or more needles for subcutaneous injection to a patient in need thereof, wherein said reservoir contains a therapeutically effective amount of the ultra fast-acting subcutaneously injectable insulin formulation of claim 1.

66. (canceled)

67. A bimodal injection system for subcutaneous injection of insulin and glucagon as needed by a patient, the system comprising a first reservoir and a second reservoir, each communicating with one or more needles for subcutaneous injection to a patient in need thereof, wherein the first reservoir contains a therapeutically effective amount of the ultra fast-acting subcutaneously injectable insulin formulation of claim 1 and the second reservoir contains a therapeutically effective amount of a subcutaneously injectable glucagon formulation.

68. A method of treating a patient in need of insulin comprising subcutaneously administering to the patient in need thereof a therapeutically effective amount of the ultra fast-acting subcutaneously injectable insulin formulation of claim 1.

69. The method of claim 68, wherein the subcutaneous administration is made from an insulin pump, insulin pen, syringe with needle, or insulin patch pump, or microneedle pump.

70-78. (canceled)

79. A method of treating diabetes mellitus in a patient in need thereof, comprising administering to the patent the formulation of claim 1.

80. The method of claim 78, wherein the diabetes mellitus is any one of Type 2 diabetes, Type 1 diabetes and gestational diabetes.

81. A method of treating hypoglycemia in a patient in need thereof, comprising administering to the patent the formulation of claim 1.

82-85. (canceled)
